(19) United States
(12) Patent Application Publication
(10) Pub. No.: US 2015/0343008 A1
(43) Pub. Date: Dec. 3, 2015

(54) Homeopathic formulation useful for treating neurological disorders

(71) Applicants: GUNVANT DEVICHAND OSWAL, Pune (IN); POOJA GUNWANT OSWAL, Pune (IN)

(72) Inventors: GUNVANT DEVICHAND OSWAL, Pune (IN); POOJA GUNWANT OSWAL, Pune (IN)

(21) Appl. No.: 14/811,858

(22) Filed: Jul. 29, 2015

Related U.S. Application Data
(63) Continuation-in-part of application No. 12/989,113, filed on Oct. 22, 2010.

(51) Int. Cl. A61K 36/81; A61K 33/14; A61K 33/00; A61K 33/42; A61K 36/28; A61K 36/185

(52) U.S. Cl. CPC A61K 36/81; A61K 36/28; A61K 36/185; A61K 33/00; A61K 33/42; A61K 33/14

(57) ABSTRACT

The present invention relates to a homeopathic formulation useful for treating neurological disorders.

### CROSS REFERENCES TO RELATED APPLICATIONS

This application claims benefit under 35 U.S.C. 120 of the previously filed Non-provisional application Ser. No. 12/989,113 filed on Apr. 27, 2009 entitled “Homeopathic Formulation ” the contents of which are entirely incorporated herein by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD OF THE PRESENT INVENTION

The present invention relates to a homeopathic formulation. Particularly, the present invention relates to a homeopathic formulation for healing of neurological disorders.

### BACKGROUND OF THE INVENTION AND PRIOR ART

Neurological disorders including mental subnormality affect between 1-3% of the total worldwide population according to World Health Organization (WHO) estimates. Rehabilitation of patients with these disorders mainly depends upon measures like assistive technology; speech therapy; behavioral therapy; occupational therapy; counselling; symptomatic drug therapy for convulsions, involuntary movements, spasticity, etc; and surgery for corrective measures. But currently there is very little choice of treatment to heal these disorders and they are generally considered irreversible. The development of an effective medical and drug-based treatment of these disorders is thus a challenge before the medical field.

The history of homeopathy dates back to the eighteenth century and the research of the German physician Samuel Hahnemann, who postulated the principle of “like cures like”. In the nineteenth century, Hugo Paul Friedrich Schultz postulated that toxins can have the opposite effect in small doses compared to large doses. In 1888, Schultz showed that very low concentrations of yeast toxins increased yeast growth over 100 fold. At the same time, the psychiatrist Rudolph Arndt developed his “Basic Law of Biology,” which states that weak stimuli slightly accelerate the vital activity, middle-strong stimuli raise it, strong stimuli suppresses it, and very strong stimuli halt vital activity. These separate observations were formulated by Arndt in 1888 into one of the earliest laws of pharmacology representing the homeopathic effect, the Arndt-Schultz rule, which states: every stimulus on a living cell elicits an activity, which is inversely proportional to the intensity of the stimulus (Martius F., 1923, Das Arndt-Schultz Grundgesetz, Muench Med. Wschr., 70(31):1005-1006). This law was later restated by Ferdinand Hueppe as: for every substance, small doses stimulate, moderate doses inhibit, large doses kill.

Allopathic medicine, with its emphasis on moderate drug doses, works to inhibit undesired physical symptoms and to kill undesired pathogens. Homeopathic medicine, on the other hand, begins with small doses and moves towards progressively higher dilutions to stimulate the body's own natural electromagnetic forces. One of the basic tenets of homeopathic medicine is that a cure for a disease can be evoked by using a high dilution medicine that resembles, yet is different from, the cause of the disease.

Critical reviews of more than 100 controlled and/or clinical studies of homeopathy show that patients received positive healing benefits from homeopathy beyond the placebo effect e.g. Jonas et al, 2003, Ann. Intern. Med. 138:393-399; Linde et al, 1997, Lancet. 350(9081):834-843; Reilly et al, 1994, Lancet. 344:1601-1606); Kleijnen et al, 1991, Bmj. 910418 302(6772):316-323. Homeopathy is widely accepted as a useful therapeutic approach throughout Europe, N. America, the British Commonwealth countries, and India.

Ayurveda mentions the effectiveness of minute doses of medicine in treating ailments. The word Ayurveda is made up of two basic terms viz. “AYU” and “VEDA” wherein “AYU” stands for life and “VEDA” means science or knowledge. Thus Ayurveda means “the science of life”.

Ayurveda says there is not a single substance in this Universe which has no medicinal value. A “VAIDYA” (conventional name for a Doctor in Ayurveda) has to use his/her intelligence and keep on preparing newer combinations of remedies, i.e. “KALPA” to bring a state of health in patients or sufferers. Ayurveda says a substance works in the body by its properties i.e. “GUNA”. The medicinal properties of a substance can be increased by subjecting it to various treatments i.e. “SANSKAR”. Due to “SANSKAR”, medicinal properties i.e. “GUNA” can be increased or decreased; sometimes “SANSKAR” can also alter the site of action of these substances inside the body. By subjecting the medicine to different treatments i.e. “SANSKARS”, the medicine can be administered in minute doses i.e. “SOOKSHMA”, which can even increase its effectiveness, while reducing the chances of side effects. A medicine is called “SOOKSHMA” when it goes deep inside the body, or because of its fine size it enters the smallest “SROTASAS”, which are the channels of circulation or tracts within the body. “SOOKSHMA” means minute quantity and minute size, and being so, it can be absorbed into the “SROTASAS” faster. “SROTASAS” are named so because of their tendency to trickle or ooze (“SRU” means flow) secretions through them (these secretions may be correlated with neurotransmitters). “SROTASAS” are the pathways for the nutrient products, waste products and “DOSHAS” during the process of metabolism (“DOSHAS” are explained in Ayurveda as entities, which when in equilibrium, the body is in good health; and when their equilibrium is disturbed, pathologies are produced in the body). While the basic sites of “SROTASAS” with different functions are fixed, their openings are innumerable, (these openings can be compared to connection of neurons at synapses.) A medicine is called “VYAVAYI”, when a medicine before being assimilated in the gastrointestinal tract gets circulated in the complete body and then it is digested. Because of “VYAVAYI” property, a medicine is absorbed instantaneously and it goes to the minutest “SROTASAS” immediately and it acts immediately. A medicine is called “AASHUKARI” when after administration it spreads in whole body immediately and acts quickly.

According to basic principles of pharmacology in Ayurveda, when two medicinal substances having similar properties, i.e. “GUNAS” are combined together, it increases their potential (Synergism). A medicine should be given in such a manner that it will be pleasant to the mind. A medicine is ideal if it can be given in many ways and has many properties, i.e. “GUNAS”. An ideal medicine should have following properties:
a. It should be effective in low dose
b. It should have immediate action
c. It should be able to act on various pathologies (“DOSHAS”)
d. It should be easily assimilable
e. It should be tasty
f. It should be pleasant
g. It should be able to cure the disease or pathology or improve the condition

According to Ayurveda, the treatment of neurological conditions should preferably involve ingredients which act as a gentle counterbalance to the neurological symptoms, i.e. “SHAMANA CHIKITSA”. This is done with the help of specific plants and body minerals that have action on neurological symptoms and conditions.

A number of homeopathic formulations have been developed in the prior art such as U.S. Pat. No. 7,229,648 which discloses a homeopathic formulation useful for treating pain and/or inflammation. Again, U.S. Pat. No. 7,037,532 discloses a hangover relief composition comprising a mixture of aqueous ethanolic tinctures of six or seven homeopathic ingredients. However, these formulations are not intended to be used for treating neurological disorders.

United States Patent Application Publication 2006/0088575 discloses homeopathic preparations of purified growth factors including Nerve Growth Factor (NGF) and purified growth hormones and associated carriers. Although the preparation is suitable for treating illnesses such as chronically injured sensory afferent nerves in adult spinal cord, olfactory defects and sensory regression, microglial deactivation and the like, the preparation is not meant for the treatment of neurological conditions such as Cerebral Palsy, Mental Subnormality, Autism, Down's Syndrome, Global Delays, Developmental Disabilities, Neuropathies, Brain Injuries, various Neurological Syndromes, Neurometabolic Disorders, Stroke, and the like.

Therefore, there is felt a need for an inexpensive homeopathic formulation which can be effectively used for the treatment of neurological disorders including those mentioned above.

### OBJECTS OF THE INVENTION

An object of the present invention is to provide a homeopathic formulation for healing of neurological disorders.

Another object of the present invention is to provide a homeopathic formulation which is effective against several types of neurological disorders including, but not limited to Cerebral Palsy—spastic, hyptonic, athetoid, ataxic; Mental Subnormality; Depression arising from neurological disorders; Autism; Down Syndrome; Global Delays; Developmental Disabilities; Neuropathies; Brain Injuries; Neurometabolic Disorders; Stroke; Attention deficit disorder (ADD); Attention deficit hyperactivity disorder (ADHD); Apert's Syndrome; Congenital Brain Anomalies like Schizencephaly, Pachygyria, Porencephaly; Dystonias; Herido-Familial Mental Retardation; Infantile Hemiplegia; Kernicterus; Learning Disabilities; Multi-Infarct Dementias; Microcephaly; Metabolic Disorders with Neurological Complications; Post Meningo-Encephalitic Brain Damage; Rubinstein Taybi Syndrome; Retts Syndrome; Post Surgical Neurodeficit; Multiple Sclerosis; Subacute sclerosing panencephalitis (SSPE) and various Syndromes and conditions of developmental delay and neurological conditions like Leigh's Encephalopathy, Cornelia De Lange Syndrome, Japanese Encephalitis, Tuberous Sclerosis, Leukodystrophy, Chromosomal Anamolies, Agenesis of Corpus Callosum, and Spinocerebellar Syndrome

Yet another object of the present invention is to provide a homeopathic formulation which is stable.

Still another object of the present invention is to provide a homeopathic formulation which is non-toxic.

Still another object of the present invention is to provide a homeopathic formulation which is free of any side effects.

Still another object of the present invention is to provide a homeopathic formulation which is easy to prepare.

Still another object of the present invention is to provide a homeopathic formulation which can be combined with other homeopathic preparations to achieve desirable effects.

Still another object of the present invention is to provide a homeopathic formulation which is compatible with conventional methods of treatment and other therapies.

Still another object of the present invention is to provide a homeopathic formulation which is cost-effective

### STATEMENT OF THE INVENTION

In accordance with the present invention, there is provided a formulation for healing neurological disorders, said formulation comprising, along with physiologically acceptable carriers, tinctures and/or homeopathic preparations of:
a) at least one ingredient selected from a first group consisting of Allium cepa, Arnica montana, Bellis perennis, Calendula officinalis, Hypericum perforatum, and Phosphoricum acidum;
b) at least one ingredient selected from a second group consisting of Aconitum napellus, Alumina, Anahalonium lewinii, Argentum nitricum, Arnica montana, Arsenicum album, Belladonna, Caulophyllum thalictroides, Causticum, Cocculus indicus, Conium maculatum, Cuprum metallicum, Curare, Dulcamara, Formica rufa, Gelsemium sempervirens, Hypericum perforatum, Kalium iodatum, Kalium tartaricum, Kalmia latifolia, Lachesis mutus, Lathyrus sativus, Latrodectus hasselti, Manganum aceticum, Mercurius corrosivus, Nux vomica, Oxalicum acidum, Phosphorus, Physostigma venenosum, Picricum acidum, Plumbum aceticum, Rhus toxicodendron, Secale cornutum, Strychninum purum, Thaelium metallicum, and Thyroidinum;
c) at least one ingredient selected from a third group consisting of Argentum nitricum, Atropinum, Aurum metallicum, Baryta carbonica, Belladonna, Calcarea carbonica, Causticum, Chelidonium majus, Crotalus horridus, Gelsemium sempervirens, Lathyrus sativus, Lycopodium clavatum, Nux vomica, Oxalicum acidum, Phosphorus, Physostigma venenosum, Plumbum metallicum, Silicea terra, Strychninum purum, Sulphur, Tarentula hispanica, and Thuja occidentalis;
d) at least one ingredient selected from a fourth group consisting of Baryta carbonica, Calcarea carbonica, Calcarea phosphorica, Causticum, Natrium muriaticum, and Silicea terra;
e) at least one ingredient selected from a fifth group consisting of Absinthium, Aconitum napellus, Aethusa cynapium, Agnus castus, Alumina, Ambra grisea, Anacardium orientale, Anhalonium lewinii, Argentum nitricum, Arnica montana, Aurum metallicum, Azadirachta indica, Baryta carbonica, Calendula officinalis, Calcarea carbonica, Calcarea phoshphorica, Camphora officinalis, Cannabis indica, Carbo vegitabilis, Cocculus indicus, Conium maculatum, Glycerinum, lchthyolum, Kalium Bromatum, Kalium carbonicum, Kalium phosphoricum, Lac caninum, Lachesis mutus, Lecithinum, Lycopodium clavatum, Medorrhinum, Mercurius solubilis—Hydrargyrum, Natrium carbonicum, Natrium muriaticum, Nitricum acidum, Nux moschata, Nux vomica, Oleander—Nerium odorum, Opium—Papaver somniferum, Phosphoricum acidum, Phosphorus, Picricum acidum, Plumbum metallicum, Rhododendron chrysanthum, Rhus toxicodendron, Selenium metallicum, Sepia officinalis, Silicea terra, Sulphur, Syphilinum, Tellurium metallicum, Thyroidinum, Zincum metallicum, Zincum phosphoricum, and Zincum picricum;
f) at least one ingredient selected from a sixth group consisting of Aesculus glabra, Agaricus muscarius, Anacardium orientale, Anhalonium lewinii, Atropninum, Baryta carbonica, Belladonna, Bothrops Lanciolatus, Bovista lycoperdon, Bufo rana, Cannabis indica, Cannabis sativa, Causticum, Cereus serpentinus, Cicuta virosa, Cuprum metallicum, Gelsemium sempervirens, Hyoscyamus niger, Ignatia amara, Kalium bromatum, Kalium cyanatum, Lachesis mutus, Laurocerasus, Mercurius solubilis—Hydrargyrum, Mygale lasiodora, Naja tripudians, Natrium muriaticum, Nux moschata, Oleander—Nerium odorum, Opium—Papaver somniferum, Phosphorus, Stramonium, Sulfonalum, Thuja occidentalis, and Vipera berus;
g) at least one ingredient selected from a seventh group consisting of Aethusa cynapium, Ailanthus glandulosa, Alfalfa, Anacardium orientale, Anhalonium lewinii, Argentum nitricum, Avena sativa, Baptisia tinctoria, Calcarea carbonica, Calcarea phosphorica, Coca—Erythroxylon coca, Cocculus indicus, Cuprum metallicum, Gelsemium sempervirens, Kalium bromatum, Kalium phosphoricum, Lecithinum, Natrium muriaticum, Nux vomica, Phosphoricum acidum, Phosphorus, Picricum acidum, Silicea terra, Stychninum phosphoricum, Zincum metallicum, Zincum phosphoricum, and Zincum picricum;
h) at least one ingredient selected from an eighth group consisting of Belladonna, Bufo rana, Cantharis vesicatoria, Cuprum metallicum, Hyoscyamus niger, Lilium tigrinum, Secale cornutum, Stramonium, Tarentula hispanica, and Veratrum album; and
i) at least one ingredient selected from a ninth group consisting of Aconitum napellus, Agaricus muscarius, Apis mellifica, Argentum nitricum, Arnica montana, Arsenicum album, Atropinum, Belladonna, Benzoicum acidum, Calcarea carbonica, Cantharis vesicatoria, Causticum, Cicuta virosa, Cimicifuga racemosa, Cina maritima, Conium maculatum, Dulcamara, Equisetum hyemale, Eryngium aquaticum, Eupatorium perfoliatum, Eupatorium purpureum, Ferrum metallicum, Ferrum phosphoricum, Gelsemium sempervirens, Hydrangea arborescens, Hyoscyamus niger, Kalium bromatum, Kalium nitricum, Kalium phosphoricum, Kreosotum, Linaria vulgaris, Lupulus humulus, Lycopodium clavatum, Magnasia phosphorica, Medorrhinum, Nux vomica, Opium—Papaver somniferum, Petroleum, Phosphoricum acidum, Physostigma venenosum, Plantago major, Pulsatilla pratensis, Rhus aromatica, Rhus toxicodendron, Sabal serrulata, Sanicula aqua, Santoninum, Secale cornutum, Senega, Sepia officinalis, Silicea terra, Stramonium, Sulphur, Terebinthiniae oleum, Thyroidinum, Thuja occidentalis, Triticum repens—Agropyrum repens, Tuberculinum bovinum Kent, Uranium nitricum, Verbascum thapsus, and Zincum metallicum.

Typically, the formulation is effective in healing neurological disorders in human beings and may also have a positive effect in neurological disorders in animals.

Typically, the ingredients are in the form of tincture.

Preferably, each of the ingredients is potentized.

Typically, the ingredients are in the form of aqueous extracts.

Preferably, the ingredients are in the form of ethanolic solutions.

Typically, the ingredients have a potency ranging from tincture to all X, C and LM (1:50,000 dilution ratio) potencies and above, preferably ranging from about 30 C to about 1 M.

Typically, the potencies may either be ‘X’ potencies or ‘C’ potencies.

Typically, the physiologically acceptable carriers are selected from a group consisting of whey, sucrose, calcium carbonate, microcrystalline cellulose, carbon, carnauba wax, croscarmellose sodium, stearic acid, magnesium stearate, silicon dioxide and ethanol.

Typically, the proportion of each of the ingredients of one group ranges from about 1:1 to about 1:10 with respect to member of the other group.

Typically, the formulation is in a form selected from the group consisting of tablets, capsules, powders, globules, lozenges, pills, pellets, solution, syrup, elixir, suspension and emulsion.

In accordance with a preferred embodiment of the present invention, there is provided a formulation for healing neurological disorders, said formulation comprising, along with physiologically acceptable carriers, the following ingredients:
i) Arnica montana;
ii) Hypericum perforatum;
iii) Causticum;
iv) Hyoscyamus niger;
v) Zincum phosphoricum;
vi) Natrium muriaticum;
vii) Calcarea phosphorica;
viii) Kalium phosphoricum; and
ix) Ferrum phosphoricum.

In still another embodiment of the present invention, there is provided a formulation comprising, along with physiologically acceptable carriers, Bellis perennis, Calcarea carbonica, Alfalfa, Avena sativa, Zincum metallicum, Stramonium, Causticum; Natrium muriaticum, and Kalium phosphoricum preferably at a potency ranging from 30 C to 1 M.

In accordance with still another aspect of the present invention, there is provided a method of making a formulation for healing neurological disorders, said method comprising the following steps:
i) selecting at least one ingredient from the first group as mentioned above;
   a. at least one ingredient from the second group as mentioned above;
   b. at least one ingredient from the third group as mentioned above;
   c. at least one ingredient from the fourth group as mentioned above;
   d. at least one ingredient from the fifth group as mentioned above;
   e. at least one ingredient from the sixth group as mentioned above;
   f. at least one ingredient from the seventh group as mentioned above;
   g. at least one ingredient from the eighth group as mentioned above;
   h. at least one ingredient from the ninth group as mentioned above;
ii) making a mixture containing selected ingredients at a ratio ranging from about 1:1 to about 1:10 with respect to member of the other group;
iii) diluting the mixture with a liquid carrier selected from water or ethanol to obtain a mixture having potency between tincture and LM potencies, preferably ranging from about 30 C to about 1 M; and
iv) soaking carrier globules in the diluted mixture to obtain said formulation.

In yet another aspect of the present invention, the individual ingredients may be potentized separately and then pre-determined levels of the potentized solution may be mixed together to obtain the homeopathic formulation which can then be soaked into carrier globules.

Alternatively, the formulations of the individual ingredients can also be obtained from commercial sources and then added to the carrier globules.

Typically, the carrier globules are sucrose globules or lactose globules.

In yet another aspect of the present invention, there is provided a method of treating neurological disorders in a subject, said method comprising administering a potentized amount of a formulation comprising, along with physiologically acceptable carriers, extracts of:
a) at least one ingredient selected from a first group consisting of Allium cepa, Arnica montana, Bellis perennis, Calendula officinalis, Hypericum perforatum, and Phosphoricum acidum;
b) at least one ingredient selected from a second group consisting of Aconitum napellus, Alumina, Anahalonium lewinii, Argentum nitricum, Arnica montana, Arsenicum album, Belladonna, Caulophyllum thalictroides, Causticum, Cocculus indicus, Conium maculatum, Cuprum metallicum, Curare, Dulcamara, Formica rufa, Gelsemium sempervirens, Hypericum perforatum, Kalium iodatum, Kalium tartaricum, Kalmia latifolia, Lachesis mutus, Lathyrus sativus, Latrodectus hasselti, Manganum aceticum, Mercurius corrosivus, Nux vomica, Oxalicum acidum, Phosphorus, Physostigma venenosum, Picricum acidum, Plumbum aceticum, Rhus toxicodendron, Secale cornutum, Strychninum purum, Thaelium metallicum, and Thyroidinum;
c) at least one ingredient selected from a third group consisting of Argentum nitricum, Atropinum, Aurum metallicum, Baryta carbonica, Belladonna, Calcarea carbonica, Causticum, Chelidonium majus, Crotalus horridus, Gelsemium sempervirens, Lathyrus sativus, Lycopodium clavatum, Nux vomica, Oxalicum acidum, Phosphorus, Physostigma venenosum, Plumbum metallicum, Silicea terra, Strychninum purum, Sulphur, Tarentula hispanica, and Thuja occidentalis;
d) at least one ingredient selected from a fourth group consisting of Baryta carbonica, Calcarea carbonica, Calcarea phosphorica, Causticum, Natrium muriaticum, and Silicea terra;
e) at least one ingredient selected from a fifth group consisting of Absinthium, Aconitum napellus, Aethusa cynapium, Agnus castus, Alumina, Ambra grisea, Anacardium orientale, Anhalonium lewinii, Argentum nitricum, Arnica montana, Aurum metallicum, Azadirachta indica, Baryta carbonica, Calendula officinalis, Calcarea carbonica, Calcarea phoshphorica, Camphora officinalis, Cannabis indica, Carbo vegitabilis, Cocculus indicus, Conium maculatum, Glycerinum, lchthyolum, Kalium Bromatum, Kalium carbonicum, Kalium phosphoricum, Lac caninum, Lachesis mutus, Lecithinum, Lycopodium clavatum, Medorrhinum, Mercurius solubilis—Hydrargyrum, Natrium carbonicum, Natrium muriaticum, Nitricum acidum, Nux moschata, Nux vomica, Oleander—Nerium odorum, Opium—Papaver somniferum, Phosphoricum acidum, Phosphorus, Picricum acidum, Plumbum metallicum, Rhododendron chrysanthum, Rhus toxicodendron, Selenium metallicum, Sepia officinalis, Silicea terra, Sulphur, Syphilinum, Tellurium metallicum, Thyroidinum, Zincum metallicum, Zincum phosphoricum, and Zincum picricum;
f) at least one ingredient selected from a sixth group consisting of Aesculus glabra, Agaricus muscarius, Anacardium orientale, Anhalonium lewinii, Atropninum, Baryta carbonica, Belladonna, Bothrops Lanciolatus, Bovista lycoperdon, Bufo rana, Cannabis indica, Cannabis sativa, Causticum, Cereus serpentinus, Cicuta virosa, Cuprum metallicum, Gelsemium sempervirens, Hyoscyamus niger, Ignatia amara, Kalium bromatum, Kalium cyanatum, Lachesis mutus, Laurocerasus, Mercurius solubilis—Hydrargyrum, Mygale lasiodora, Naja tripudians, Natrium muriaticum, Nux moschata, Oleander—Nerium odorum, Opium—Papaver somniferum, Phosphorus, Stramonium, Sulfonalum, Thuja occidentalis, and Vipera berus;
g) at least one ingredient selected from a seventh group consisting of Aethusa cynapium, Ailanthus glandulosa, Alfalfa, Anacardium orientale, Anhalonium lewinii, Argentum nitricum, Avena sativa, Baptisia tinctoria, Calcarea carbonica, Calcarea phosphorica, Coca—Erythroxylon coca, Cocculus indicus, Cuprum metallicum, Gelsemium sempervirens, Kalium bromatum, Kalium phosphoricum, Lecithinum, Natrium muriaticum, Nux vomica, Phosphoricum acidum, Phosphorus, Picricum acidum, Silicea terra, Stychninum phosphoricum, Zincum metallicum, Zincum phosphoricum, and Zincum picricum;
h) at least one ingredient selected from an eighth group consisting of Belladonna, Bufo rana, Cantharis vesicatoria, Cuprum metallicum, Hyoscyamus niger, Lilium tigrinum, Secale cornutum, Stramonium, Tarentula hispanica, and Veratrum album, and
i) at least one ingredient selected from a ninth group consisting of Aconitum napellus, Agaricus muscarius, Apis mellifica, Argentum nitricum, Arnica montana, Arsenicum album, Atropinum, Belladonna, Benzoicum acidum, Calcarea carbonica, Cantharis vesicatoria, Causticum, Cicuta virosa, Cimicifuga racemosa, Cina maritima, Conium maculatum, Dulcamara, Equisetum hyemale, Eryngium aquaticum, Eupatorium perfoliatum, Eupatorium purpureum, Ferrum metallicum, Ferrum phosphoricum, Gelsemium sempervirens, Hydrangea arborescens, Hyoscyamus niger, Kalium bromatum, Kalium nitricum, Kalium phosphoricum, Kreosotum, Linaria vulgaris, Lupulus humulus, Lycopodium clavatum, Magnasia phosphorica, Medorrhinum, Nux vomica, Opium—Papaver somniferum, Petroleum, Phosphoricum acidum, Physostigma venenosum, Plantago major, Pulsatilla pratensis, Rhus aromatica, Rhus toxicodendron, Sabal serrulata, Sanicula aqua, Santoninum, Secale cornutum, Senega, Sepia officinalis, Silicea terra, Stramonium, Sulphur, Terebinthiniae oleum, Thyroidinum, Thuja occidentalis, Triticum repens—Agropyrum repens, Tuberculinum bovinum Kent, Uranium nitricum, Verbascum thapsus, and Zincum metallicum

Typically, the homeopathic formulation is effective against several types of neurological disorders including, but not limited to Cerebral Palsy—spastic, hyptonic, athetoid, ataxic; Mental Subnormality; Depression arising from neurological disorders; Autism; Down Syndrome; Global Delays; Developmental Disabilities; Neuropathies; Brain Injuries; Neurometabolic Disorders; Stroke; Attention deficit disorder (ADD); Attention deficit hyperactivity disorder (ADHD); Apert's Syndrome; Congenital Brain Anomalies like Schizencephaly, Pachygyria, Porencephaly; Dystonias; Herido-Familial Mental Retardation; Infantile Hemiplegia; Kernicterus; Learning Disabilities; Multi-Infarct Dementias; Microcephaly; Metabolic Disorders with Neurological Complications; Post Meningo-Encephalitic Brain Damage; Rubinstein Taybi Syndrome; Retts Syndrome; Post Surgical Neurodeficit; Multiple Sclerosis; Subacute sclerosing panencephalitis (SSPE) and various Syndromes and conditions of developmental delay and neurological conditions like Leigh's Encephalopathy, Cornelia De Lange Syndrome, Japanese Encephalitis, Tuberous Sclerosis, Leukodystrophy, Chromosomal Anamolies, Agenesis of Corpus Callosum, and Spinocerebellar Syndrome.

In accordance with the present invention, there is provided a formulation based on the principle of synergistic effects of various homeopathic medicines that are complementary to each other, and taken together produce holistic healing and a unique treatment of various neurological disorders.

The development of the present invention is based on homeopathic principles. The potentized dilutions in the combination of the present invention act in small doses, and hence stimulate, according to Ferdinand Hueppe's law: for every substance, small doses stimulate, moderate doses inhibit, large doses kill.

The potentized dilutions in the formulation of the present invention act on the principle of “like cures like” and/or act as catalysts to speed up the recovery. It is hypothesized that the ingredients of the present invention such as Natrium Muriaticum (i.e. Sodium), Calcerea Phosphorica (i.e. Calcium) and Kalium Phosphoricum (i.e. Potassium) take care of action potentials which are required for nerve impulse transmission. It is also hypothesized that the homeopathically prepared minerals in the present invention restore the disturbed molecular motions of the minerals to their normal state. Also, it is hypothesized that the minerals in the present invention reactivate the chemical changes for neurotransmission, while the herbal extracts act as a catalyst to speed up the improvement. Possibly, the present invention removes the blockage of synaptic levels or helps myelination. The ingredients of the present invention are hypothesized to improve the iron and haemoglobin levels of the blood, thus supplying more oxygen to the tissues and cells of the body leading to improved metabolism and immunity of the patient.

The development of the present invention incidentally also finds echoes in the principles of Ayurveda as described above—for example, the combination has synergistic effect, it is prepared using a new combinations of remedies, (“KALPA”), is given in low dosage (“SOOKSHMA”), has an immediate action even before being assimilated in the gastrointestinal tract (“VYAVAYI”), spreads in the whole body immediately (“ASHUKARI”), is pleasant to take, and acts on various pathologies.

In this light, the present invention discloses the effectiveness of the formulation in a number of cases of neurological disorders such Cerebral Palsy, Mental Subnormality, Autism, Down's Syndrome, Global Delays, Developmental Disabilities, Neuropathies, Brain Injuries, various Neurological Syndromes, Neurometabolic Disorders, Stroke, and the like, and therefore represents a breakthrough in the medical field.

### DETAILED DESCRIPTION OF THE INVENTION

The description thereto is merely illustrative and only exemplifies the present invention and in no way limits the scope thereof.

In this patent specification, the terms “tincture” and “homeopathic preparation” of an ingredient refer to extracts of a part, combinations of parts and/or the entirety of the ingredient. The “tincture” can be prepared by exposing a part; parts and/or the entirety of the ingredient in a solvent, e.g. alcohol and/or water. The “tincture” of an ingredient preferably is a mother tincture of the ingredient prepared according to the procedures in the Homeopathic Pharmacopeia of the United States (H PUS). The “homeopathic preparation” can be prepared by dilution of the “tincture” with an appropriate liquid such as water or alcohol. The “homeopathic preparation” of an ingredient for the formulation of the invention is preferably prepared as per HPUS procedures, wherein the mother tincture of the ingredient is serially diluted and subjected to succussion according to the target potency using potentization procedures known in the art of homeopathy.

Further, the term “potency” of a homeopathic remedy refers to how many times it has undergone the process of serial dilution and succussion (known as “potentization”), and therefore how far it has been removed from a crude or material form. This process is carried out according to a number of different scales: Decimal, Centessimal and Fifty-Millessimal.

Decimal scale (1:10): Referred to as “D Potencies” (for “Decimal”), or “X Potencies” after the Roman numeral. One part of the original liquid substance is added to 9 parts of a carrier such as alcohol, and succussed (shaken vigorously) 10 times. The resulting product is referred to as a “D1” or “1×”. One part of this is then added to 9 parts of alcohol and succussed 10 times, resulting in a D2 (2.times.) and so on. A medicine subjected to this process 30 times will thus be called a D30 (30.times.).

For potentisation of non-liquid substances, the process is done using a carrier of milk-sugar rather than alcohol, and triturated rather than succussed. Once a dilution ratio of 1:1,000,000 (6.times. or D6) has been reached, the insoluble substance is then rendered soluble, and preparation can be continued in a liquid medium as described above.

Centessimal scale (1:100): Referred to as “C Potencies” (for “Centessimal”). One part of the original liquid substance is added to 99 parts of a carrier such as alcohol, and succussed (shaken vigorously) 10 times. The resulting product is referred to as a “C1” or “1 C”. One part of this is then added to 99 parts of alcohol and succussed 10 times, resulting in a 2 C and so on. A medicine subjected to this process 30 times will thus be called 30 C, 200 times will be called 200 C and so on. For the sake of ease in labeling, at higher potencies the numbers are dropped in favour of Roman numerals. For example 1000 C is shortened to 1 M (M=Millessimal, or 1000), 10,000 C=10 M, 50,000 C=50 M, 100,000 C=CM, 1,000,0000=MM and the like.

For potentisation of non-liquid substances, the process is done using a carrier of milk-sugar rather than alcohol, and triturated rather than succussed. Once a dilution ratio of 1:1,000,000 (3 C) has been reached, the insoluble substance is then rendered soluble, and preparation can be continued in a liquid medium as described above.

Fifty-Millessimal scale (1:50,000): Referred to as “Q Potencies” (Q stands for quinquagintamillesimal), they are also called “LM Potencies” after the Roman numerals, although this is an incorrect use of Roman numerals (LM actually means 950, not 50,000).

The term ‘succussion’ means vigorous shaking of a diluted homeopathic preparation in order to activate the medicinal substance.

Trituration is the name of the process for reducing the particle size of a substance by grinding, as by grinding of powders in a mortar with a pestle.

The present invention discloses a combination of homeopathic medicines for the treatment of neurological disorders and conditions including, but not limited to Cerebral Palsy—spastic, hyptonic, athetoid, ataxic; Mental Subnormality; Depression arising from neurological disorders; Autism; Down Syndrome; Global Delays; Developmental Disabilities; Neuropathies; Brain Injuries; Neurometabolic Disorders; Stroke; Attention deficit disorder (ADD); Attention deficit hyperactivity disorder (ADHD); Apert's Syndrome; Congenital Brain Anomalies like Schizencephaly, Pachygyria, Porencephaly; Dystonias; Herido-Familial Mental Retardation; Infantile Hemiplegia; Kernicterus; Learning Disabilities; Multi-Infarct Dementias; Microcephaly; Metabolic Disorders with Neurological Complications; Post Meningo-Encephalitic Brain Damage; Rubinstein Taybi Syndrome; Retts Syndrome; Post Surgical Neurodeficit; Multiple Sclerosis; Subacute sclerosing panencephalitis (SSPE) and various Syndromes and conditions of developmental delay and neurological conditions like Leigh's Encephalopathy, Cornelia De Lange Syndrome, Japanese Encephalitis, Tuberous Sclerosis, Leukodystrophy, Chromosomal Anamolies, Agenesis of Corpus Callosum, and Spinocerebellar Syndrome.

The present invention also has positive effects on the mood and reduces mental depression. Further, the present invention also improves the texture of the skin. Improvements are also seen in genetic and metabolic disorders.

The present invention relates to a formulation for healing neurological disorders, said formulation comprising, along with physiologically acceptable carriers, the following ingredients:
a) at least one ingredient selected from a first group consisting of Allium cepa, Arnica montana, Bellis perennis, Calendula officinalis, Hypericum perforatum, and Phosphoricum acidum;
b) at least one ingredient selected from a second group consisting of Aconitum napellus, Alumina, Anahalonium lewinii, Argentum nitricum, Arnica montana, Arsenicum album, Belladonna, Caulophyllum thalictroides, Causticum, Cocculus indicus, Conium maculatum, Cuprum metallicum, Curare, Dulcamara, Formica rufa, Gelsemium sempervirens, Hypericum perforatum, Kalium iodatum, Kalium tartaricum, Kalmia latifolia, Lachesis mutus, Lathyrus sativus, Latrodectus hasselti, Manganum aceticum, Mercurius corrosivus, Nux vomica, Oxalicum acidum, Phosphorus, Physostigma venenosum, Picricum acidum, Plumbum aceticum, Rhus toxicodendron, Secale cornutum, Strychninum purum, Thaelium metallicum, and Thyroidinum;
c) at least one ingredient selected from a third group consisting of Argentum nitricum, Atropinum, Aurum metallicum, Baryta carbonica, Belladonna, Calcarea carbonica, Causticum, Chelidonium majus, Crotalus horridus, Gelsemium sempervirens, Lathyrus sativus, Lycopodium clavatum, Nux vomica, Oxalicum acidum, Phosphorus, Physostigma venenosum, Plumbum metallicum, Silicea terra, Strychninum purum, Sulphur, Tarentula hispanica, and Thuja occidentalis;
d) at least one ingredient selected from a fourth group consisting of Baryta carbonica, Calcarea carbonica, Calcarea phosphorica, Causticum, Natrium muriaticum, and Silicea terra;
e) at least one ingredient selected from a fifth group consisting of Absinthium, Aconitum napellus, Aethusa cynapium, Agnus castus, Alumina, Ambra grisea, Anacardium orientale, Anhalonium lewinii, Argentum nitricum, Arnica montana, Aurum metallicum, Azadirachta indica, Baryta carbonica, Calendula officinalis, Calcarea carbonica, Calcarea phoshphorica, Camphora officinalis, Cannabis indica, Carbo vegitabilis, Cocculus indicus, Conium maculatum, Glycerinum, lchthyolum, Kalium Bromatum, Kalium carbonicum, Kalium phosphoricum, Lac caninum, Lachesis mutus, Lecithinum, Lycopodium clavatum, Medorrhinum, Mercurius solubilis—Hydrargyrum, Natrium carbonicum, Natrium muriaticum, Nitricum acidum, Nux moschata, Nux vomica, Oleander—Nerium odorum, Opium—Papaver somniferum, Phosphoricum acidum, Phosphorus, Picricum acidum, Plumbum metallicum, Rhododendron chrysanthum, Rhus toxicodendron, Selenium metallicum, Sepia officinalis, Silicea terra, Sulphur, Syphilinum, Tellurium metallicum, Thyroidinum, Zincum metallicum, Zincum phosphoricum, and Zincum picricum;
f) at least one ingredient selected from a sixth group consisting of Aesculus glabra, Agaricus muscarius, Anacardium orientale, Anhalonium lewinii, Atropninum, Baryta carbonica, Belladonna, Bothrops Lanciolatus, Bovista lycoperdon, Bufo rana, Cannabis indica, Cannabis sativa, Causticum, Cereus serpentinus, Cicuta virosa, Cuprum metallicum, Gelsemium sempervirens, Hyoscyamus niger, Ignatia amara, Kalium bromatum, Kalium cyanatum, Lachesis mutus, Laurocerasus, Mercurius solubilis—Hydrargyrum, Mygale lasiodora, Naja tripudians, Natrium muriaticum, Nux moschata, Oleander—Nerium odorum, Opium—Papaver somniferum, Phosphorus, Stramonium, Sulfonalum, Thuja occidentalis, and Vipera berus;
g) at least one ingredient selected from a seventh group consisting of Aethusa cynapium, Ailanthus glandulosa, Alfalfa, Anacardium orientale, Anhalonium lewinii, Argentum nitricum, Avena sativa, Baptisia tinctoria, Calcarea carbonica, Calcarea phosphorica, Coca—Erythroxylon coca, Cocculus indicus, Cuprum metallicum, Gelsemium sempervirens, Kalium bromatum, Kalium phosphoricum, Lecithinum, Natrium muriaticum, Nux vomica, Phosphoricum acidum, Phosphorus, Picricum acidum, Silicea terra, Stychninum phosphoricum, Zincum metallicum, Zincum phosphoricum, and Zincum picricum;
h) at least one ingredient selected from an eighth group consisting of Belladonna, Bufo rana, Cantharis vesicatoria, Cuprum metallicum, Hyoscyamus niger, Lilium tigrinum, Secale cornutum, Stramonium, Tarentula hispanica, and Veratrum album; and
i) at least one ingredient selected from a ninth group consisting of Aconitum napellus, Agaricus muscarius, Apis mellifica, Argentum nitricum, Arnica montana, Arsenicum album, Atropinum, Belladonna, Benzoicum acidum, Calcarea carbonica, Cantharis vesicatoria, Causticum, Cicuta virosa, Cimicifuga racemosa, Cina maritima, Conium maculatum, Dulcamara, Equisetum hyemale, Eryngium aquaticum, Eupatorium perfoliatum, Eupatorium purpureum, Ferrum metallicum, Ferrum phosphoricum, Gelsemium sempervirens, Hydrangea arborescens, Hyoscyamus niger, Kalium bromatum, Kalium nitricum, Kalium phosphoricum, Kreosotum, Linaria vulgaris, Lupulus humulus, Lycopodium clavatum, Magnasia phosphorica, Medorrhinum, Nux vomica, Opium—Papaver somniferum, Petroleum, Phosphoricum acidum, Physostigma venenosum, Plantago major, Pulsatilla pratensis, Rhus aromatica, Rhus toxicodendron, Sabal serrulata, Sanicula aqua, Santoninum, Secale cornutum, Senega, Sepia officinalis, Silicea terra, Stramonium, Sulphur, Terebinthiniae oleum, Thyroidinum, Thuja occidentalis, Triticum repens—Agropyrum repens, Tuberculinum bovinum Kent, Uranium nitricum, Verbascum thapsus, and Zincum metallicum.

Typically, the formulation is effective in healing neurological disorders in human beings and may also have a positive effect in neurological disorders in animals.

Typically, the ingredients are in the form of tincture.

Preferably, each of the ingredients is potentized.

Typically, the ingredients are in the form of aqueous extracts.

Preferably, the ingredients are in the form of ethanolic solutions.

Typically, the ingredients have a potency ranging from tincture to all X, C and LM potencies, preferably ranging from about 30 C. to about 1 M.

Typically, the potencies may either be ‘X’ potencies or C potencies.

Typically, the physiologically acceptable carriers are selected from a group consisting of whey, sucrose, calcium carbonate, microcrystalline cellulose, carbon, carnauba wax, croscarmellose sodium, stearic acid, magnesium stearate, silicon dioxide and ethanol.

In accordance with another embodiment of the present invention, there is provided a formulation for healing neurological disorders, said formulation comprising, along with physiologically acceptable carriers, the following ingredients:
i) Arnica montana;
ii) Hypericum perforatum;
iii) Causticum;
iv) Hyoscyamus niger;
v) Zincum phosphoricum;
vi) Natrium muriaticum;
vii) Calcarea phosphorica;
viii) Kalium phosphoricum; and
ix) Ferrum phosphoricum.

In still another embodiment of the present invention, there is provided a formulation comprising, along with physiologically acceptable carriers, Bellis perennis, Calcarea carbonica, Alfalfa, Avena sativa, Zincum metallicum, Stramonium, Causticum; Natrium muriaticum, and Kalium phosphoricum at a potency ranging from 30 C to 1 M.

Typically, the proportion of each of the ingredients of one group ranges from about 1:1 to about 1:10 with respect to member of the other group.

Typically, the formulation is in a form selected from the group consisting of tablets, capsules, powders, globules, lozenges, pills, pellets, solution, syrup, elixir, suspension and emulsion.

In accordance with still another aspect of the present invention, there is provided a method of making a formulation for healing neurological disorders, said method comprising the following steps:
i) selecting at least one ingredient from the first group as mentioned above;
   a. at least one ingredient from the second group as mentioned above;
   b. at least one ingredient from the third group as mentioned above;
   c. at least one ingredient from the fourth group as mentioned above;
   d. at least one ingredient from the fifth group as mentioned above;
   e. at least one ingredient from the sixth group as mentioned above;
   f. at least one ingredient from the seventh group as mentioned above;
   g. at least one ingredient from the eighth group as mentioned above;
   h. at least one ingredient from the ninth group as mentioned above;
ii) making a mixture containing selected ingredients at a ratio ranging from about 1:1 to about 1:10 with respect to member of the other group;
iii) diluting the mixture with a liquid carrier selected from water or ethanol to obtain a mixture having potency between tincture and LM potencies, preferably ranging from about 30 C to about 1 M; and
iv) soaking carrier globules in the diluted mixture to obtain said formulation.
   In still another embodiment of the present invention, the selected ingredients at a ratio ranging from about 1:1 to about 1:10 with respect to member of the other group are first homeopathically potentized with a liquid carrier selected from water or ethanol to a potency ranging between tincture and LM potencies, preferably ranging from about 30 C to 1 M and then mixed together and soaked in carrier globules to obtain said formulation.

Typically, the carrier globules are sucrose globules or lactose globules.

In yet another embodiment of the present invention, there is provided a method of treating neurological disorders in a subject, said method comprising administering a potentized amount of a formulation comprising, along with physiologically acceptable carriers, extracts of
a) at least one ingredient selected from a first group consisting of Allium cepa, Arnica montana, Bellis perennis, Calendula off icinalis, Hypericum perforatum, and Phosphoricum acidum;
b) at least one ingredient selected from a second group consisting of Aconitum napellus, Alumina, Anahalonium lewinii, Argentum nitricum, Arnica montana, Arsenicum album, Belladonna, Caulophyllum thalictroides, Causticum, Cocculus indicus, Conium maculatum, Cuprum metallicum, Curare, Dulcamara, Formica rufa, Gelsemium sempervirens, Hypericum perforatum, Kalium iodatum, Kalium tartaricum, Kalmia latifolia, Lachesis mutus, Lathyrus sativus, Latrodectus hasselti, Manganum aceticum, Mercurius corrosivus, Nux vomica, Oxalicum acidum, Phosphorus, Physostigma venenosum, Picricum acidum, Plumbum aceticum, Rhus toxicodendron, Secale cornutum, Strychninum purum, Thaelium metallicum, and Thyroidinum;
c) at least one ingredient selected from a third group consisting of Argentum nitricum, Atropinum, Aurum metallicum, Baryta carbonica, Belladonna, Calcarea carbonica, Causticum, Chelidonium majus, Crotalus horridus, Gelsemium sempervirens, Lathyrus sativus, Lycopodium clavatum, Nux vomica, Oxalicum acidum, Phosphorus, Physostigma venenosum, Plumbum metallicum, Silicea terra, Strychninum purum, Sulphur, Tarentula hispanica, and Thuja occidentalis;
d) at least one ingredient selected from a fourth group consisting of Baryta carbonica, Calcarea carbonica, Calcarea phosphorica, Causticum, Natrium muriaticum, and Silicea terra;
e) at least one ingredient selected from a fifth group consisting of Absinthium, Aconitum napellus, Aethusa cynapium, Agnus castus, Alumina, Ambra grisea, Anacardium orientale, Anhalonium lewinii, Argentum nitricum, Arnica montana, Aurum metallicum, Azadirachta indica, Baryta carbonica, Calendula officinalis, Calcarea carbonica, Calcarea phoshphorica, Camphora officinalis, Cannabis indica, Carbo vegitabilis, Cocculus indicus, Conium maculatum, Glycerinum, lchthyolum, Kalium Bromatum, Kalium carbonicum, Kalium phosphoricum, Lac caninum, Lachesis mutus, Lecithinum, Lycopodium clavatum, Medorrhinum, Mercurius solubilis—Hydrargyrum, Natrium carbonicum, Natrium muriaticum, Nitricum acidum, Nux moschata, Nux vomica, Oleander—Nerium odorum, Opium—Papaver somniferum, Phosphoricum acidum, Phosphorus, Picricum acidum, Plumbum metallicum, Rhododendron chrysanthum, Rhus toxicodendron, Selenium metallicum, Sepia officinalis, Silicea terra, Sulphur, Syphilinum, Tellurium metallicum, Thyroidinum, Zincum metallicum, Zincum phosphoricum, and Zincum picricum;
f) at least one ingredient selected from a sixth group consisting of Aesculus glabra, Agaricus muscarius, Anacardium orientale, Anhalonium lewinii, Atropninum, Baryta carbonica, Belladonna, Bothrops Lanciolatus, Bovista lycoperdon, Bufo rana, Cannabis indica, Cannabis sativa, Causticum, Cereus serpentinus, Cicuta virosa, Cuprum metallicum, Gelsemium sempervirens, Hyoscyamus niger, Ignatia amara, Kalium bromatum, Kalium cyanatum, Lachesis mutus, Laurocerasus, Mercurius solubilis—Hydrargyrum, Mygale lasiodora, Naja tripudians, Natrium muriaticum, Nux moschata, Oleander—Nerium odorum, Opium—Papaver somniferum, Phosphorus, Stramonium, Sulfonalum, Thuja occidentalis, and Vipera berus;
g) at least one ingredient selected from a seventh group consisting of Aethusa cynapium, Ailanthus glandulosa, Alfalfa, Anacardium orientale, Anhalonium lewinii, Argentum nitricum, Avena sativa, Baptisia tinctoria, Calcarea carbonica, Calcarea phosphorica, Coca—Erythroxylon coca, Cocculus indicus, Cuprum metallicum, Gelsemium sempervirens, Kalium bromatum, Kalium phosphoricum, Lecithinum, Natrium muriaticum, Nux vomica, Phosphoricum acidum, Phosphorus, Picricum acidum, Silicea terra, Stychninum phosphoricum, Zincum metallicum, Zincum phosphoricum, and Zincum picricum;
h) at least one ingredient selected from an eighth group consisting of Belladonna, Bufo rana, Cantharis vesicatoria, Cuprum metallicum, Hyoscyamus niger, Lilium tigrinum, Secale cornutum, Stramonium, Tarentula hispanica, and Veratrum album; and
i) at least one ingredient selected from a ninth group consisting of Aconitum napellus, Agaricus muscarius, Apis mellifica, Argentum nitricum, Arnica montana, Arsenicum album, Atropinum, Belladonna, Benzoicum acidum, Calcarea carbonica, Cantharis vesicatoria, Causticum, Cicuta virosa, Cimicifuga racemosa, Cina maritima, Conium maculatum, Dulcamara, Equisetum hyemale, Eryngium aquaticum, Eupatorium perfoliatum, Eupatorium purpureum, Ferrum metallicum, Ferrum phosphoricum, Gelsemium sempervirens, Hydrangea arborescens, Hyoscyamus niger, Kalium bromatum, Kalium nitricum, Kalium phosphoricum, Kreosotum, Linaria vulgaris, Lupulus humulus, Lycopodium clavatum, Magnasia phosphorica, Medorrhinum, Nux vomica, Opium—Papaver somniferum, Petroleum, Phosphoricum acidum, Physostigma venenosum, Plantago major, Pulsatilla pratensis, Rhus aromatica, Rhus toxicodendron, Sabal serrulata, Sanicula aqua, Santoninum, Secale cornutum, Senega, Sepia officinalis, Silicea terra, Stramonium, Sulphur, Terebinthiniae oleum, Thyroidinum, Thuja occidentalis, Triticum repens—Agropyrum repens, Tuberculinum bovinum Kent, Uranium nitricum, Verbascum thapsus, and Zincum metallicum.

Typically, the ingredients disclosed in said first group act on the nervous system and aid in the healing process of Neuritis—injuries of nerves and traumatic conditions.

Typically, the ingredients disclosed in said second group act on the nervous system and aid in the healing process of Paraplegia.

Typically, the ingredients disclosed in said third group act on the nervous system and aid in the healing process of Degeneration—multiple sclerosis.

Typically, the ingredients disclosed in said fourth group act on the locomotor system and improve the Gait—walking, especially in children who are slow in learning to walk.

Typically, the ingredients disclosed in said fifth group act on the mind and enhance the memory. They may also help to regain the lost memory.

Typically, the ingredients disclosed in said sixth group act on the mind and improve the Speech. Particularly slow, difficult enunciation, inarticulate and stammering conditions can be got rid of by administration of extracts of such ingredients.

Typically, the ingredients disclosed in said seventh group act on the mind and provide relief from Brain Fag.

Typically, the ingredients disclosed in said eighth group act on the mind and mitigate the Propensity like destructive tendency, biting, striking, tearing clothes seen amongst many individuals.

Typically, the ingredients disclosed in said ninth group act on the Urinary system and heal Enuresis—Incontinence and serve as a remedy in general.

The formulation of the invention is prepared by adding the different ingredient dilutions in pre-determined quantities, typically in equal quantities to readily available tablets/globules made from lactose or sucrose. The dilutions of the homeopathic ingredients are preferably added till the tablets are thoroughly moistened. The available tablets are of various sizes. The process of preparation is explained in detail below:

A clean, dust-free reaction mixing vessel, preferably a bottle with an inert plastic cap is taken. Boiling water is poured into it so that all the air escapes out. After about 5 minutes, the water is drained out of the bottle and the bottle is air-dried until the bottle is completely de-humidified. During the process, aseptic conditions are maintained. Alternatively, the bottle can be subjected to sterilization by autoclaving.

Different pre-determined quantities of potentized ingredients are drawn from their respective containers and poured one by one into the bottle in a clean, dust-free, radiation-free and aroma-free environment. After the addition of each ingredient, the bottle is closed tightly with the cap provided and shaken vigorously either manually or by other known means for about 5-15 times, such that towards the end of the process, a homogeneous mixture is obtained.

In a separate reaction vessel (bottle), sterile sugar globules are taken such that it fills about three-fourth of the vessel. The homogeneous mixture of ingredients is slowly and uniformly poured taking care that no splashing occurs, until the liquid is completely soaked by the globules. These globules are then ready for dispensing. The bottle should be stored in air-tight, cool and aroma-free environment, away from direct U.V. light and electromagnetic radiations.

One preferred combination of the present invention comprises the herbal and non-herbal ingredients at a potency range of about 200 C.

The homeopathic ingredients of the present invention are non-toxic and do not produce undesirable side-effects. Each of the ingredients is also part of leading homeopathic pharmacopeia including the United States Homeopathic Pharmacopeia, Homeopathic Pharmacopeia of India, and the like. Some of the ingredients of the present invention are incidentally also in use in Ayurveda for many centuries, e.g. Zinc, Hyoscamus niger, and the like.

In accordance with a preferred embodiment of the present invention, the homeopathic formulation is formulated into a variety of administration forms such as oral, topical, parenteral and other novel drug delivery systems.

Oral dosage is the preferred method of delivering the formulation of the invention. The active ingredients of the homeopathic formulations may be used with any of the standard delivery systems used in oral homeopathy in any acceptable combination such as sugar pills, tablets, drops, pills, water, glycerin, milk sugar and cane sugar vehicles, alcohol, medicated powders, medicated globules (pellets, pilules), cones, etc.

The active ingredients of the homeopathic formulations may also be administered through inhalation, topical application on skin, administered parenterally, such as intravenously, or with any other method of homeopathic and/or herbal and/or allopathic drug administration, such as intramuscular, intracutaneous, intravenous or subcutaneous injections. Other possible drug delivery methods include implanting a pump or other instrument to deliver the said formulation in the body, drug delivery systems used in modern medicine, biotechnology, nanotechnology, etc. The formulation could also be taken nasally, or as eyedrops, or eardrops or in the form of a suppository or a patch. For each of these different delivery methods, the formulation would have to be of course prepared in an appropriate potency, and carried in a safe and effective base as, for example, described in OTC, HPUS and other medical literature.

Typically three pills are given in the morning and/or evening, although the quantity/dosage is relatively unimportant. It is advisable that the mouth and tongue are clean and relatively odor-free to ensure maximum absorption and availability of the formulation. Typically, three pills are first to be transferred from the bottle to the cap of the bottle or any other inert container. In this process, it is advisable that the pills should not be touched by the hands. The pills should then be placed into the mouth and chewed until they dissolve. For this, it is desirable that one should not eat or drink anything else for half an hour before and after intake of the formulation of the invention because the medicine is absorbed sub-lingually. It is also advisable that foods like non-vegetarian food and coffee and strong odor foods like raw onion or garlic should preferably be avoided to get the best results. However, it is suggested that the key effectiveness of the present invention arises from the unique combination mentioned in the formulation. The formulation of the present invention can also be given as a complement to other forms of medicine or rehabilitation.

Mechanism of Action of the Formulation

It has been postulated that in the case of homeopathy, highly dilute compounds transfer biological activity to cells by electromagnetic fields (Benveniste, 1993, Frontier Perspec. 3(2):13-15). Further it has been hypothesized by Del Giudice et al in the case of homeopathic formulations that interactions between the electric dipoles of water and the radiation fields of a charged molecule generate a permanent polarization of water that becomes coherent and has the ability to transmit specific information to cell receptors (Del Giudice, E., Preparata, G., Vitiello, G., 1988, Phys. Rev. Lett. 61:1085-1088). The positive benefits of the homeopathic method of treatment are well documented and also account for the tremendous popularity of homeopathy worldwide.

Based on the observations of patients which are described a little later in the specification, it can be suggested that the present invention is probably acting on the neurotransmitters or nerve growth factor. Also it is hypothesized that the minerals in the present invention reactivate the chemical changes for neurotransmission, while the herbal extracts act as catalysts to speed up the improvement. Possibly, the present invention removes blockage of synaptic levels or helps myelination.

None of the individual ingredients of the present invention have been described to have a treatment action on specific neurological disorders like Cerebral Palsy, Mental Subnormality, Autism, and the like. Hence, the present invention, having a widespread positive action on different neurological disorders, is indeed unique. The formulations of the present invention also do not require casework diagnostics to attempt to discover the similimum (remedy most resembling patient's symptoms) and then attempt to prescribe the correct potency, duration and dosage. Hence, the present invention is a common formulation for several neurological disorders mentioned, and no drug individualization using single drug ingredient per dose as practiced in classical homeopathy is required. All ingredients of the present invention are complementary to each other according to standard Homeopathic Pharmacopeia and the individual components are in use in Homeopathy and some in Ayurveda for many years.

While the preferred embodiment mentioned earlier might offer the best results for the aforementioned neurological disorders, it is expected that there are other possible variations of the preferred formulation of the invention that are expected to also have good therapeutic properties. The homeopathic formulations of the invention can be varied in terms of the potencies and doses, or in terms of the ingredients. The homeopathic formulation of the invention can be administered either in alcoholic or non-alcoholic form. Each of the ingredients in the formulation of the invention may also be given in potencies ranging from lower potencies including tincture to higher potencies of 50 m, LM potencies and above. The preferred homeopathic formulation of the invention comprises 9 different ingredients. Some of the formulations of the invention can also be made by not using one or more of the 9 ingredients. As one or some of the 9 ingredients are excluded from the formulations, the formulations will still have effective properties. The homeopathic formulation of the invention can comprise 8 ingredients, 7 ingredients, 6 ingredients or 5 ingredients. However, each omission of an ingredient from the list of 9 slightly lessens the total product effectiveness.

The formulation of the invention may be combined with other treatment methods and substances used in the treatment of neurological disorders including allopathic medicines, vitamins, minerals, amino acids, traditional homeopathic remedies, inert substances, etc. The individual components could be given in potentized forms and also in other forms such as, but not limited to, mother tincture, biotechnology form, nanotechnology form, etc.

The individual components of the treatment may be administered all together at the same time and/or in various permutations and combinations, for example a few ingredients could be administered together at one time, and the others at a different time; etc. Additional components could be added to the treatment that could increase its effectiveness or allow it to function with the same level of effectiveness.

The invention will now be described with the help of following example; however, this example should not be construed to limit the scope of the present invention.

### EXAMPLE 1

Potentized formulations of Arnica montana; Hypericum perforatum; Causticum; Hyoscyamus niger; Zincum phosphoricum; Natrium muriaticum; Calcarea phosphorica; Kalium phosphoricum; and Ferrum phosphoricum were obtained. These nine formulations were in potencies ranging from 30 C to 1 M. The nine formulations were first mixed together homogeneously as described above and the resulting mixture was then added to sucrose globules allowing the globules to soak the liquid, preferably until the globules were thoroughly moistened. The potentized formulations were prepared by serial dilution according to the standard homeopathic procedures.

The resultant combination formulation of the present invention was administered to twenty nine patients suffering from various neurological disorders. The details of the results and the patients that were administered are presented in the anecdotal studies below. The patients were video monitored wherever possible. Opinions about improvements by therapists and parents were recorded. Nerve conduction velocity (NCV) study was conducted whenever necessary before and after treatment.

In all these patients, before starting the invention treatment, conventional treatments like Occupational Therapy, Speech Therapy, and Symptomatic drug treatment were tried. The response seen after administration of the present invention treatment was much greater than with the previous conventional treatment indicating the efficacy of the invention. No side effects were seen in any of the patients. Following are details of improvements seen after administration of the present invention.

Anecdotal Studies:

Patient 1: A 5 year old boy was suffering from cerebral palsy spastic diplegia with a birth history of premature delivery at 7.sup.th month and having a low birth weight of 1.1 kg. He came with the following complaints: Delayed motor milestones, partial bowel and bladder control. With 2 months of treatment with the present invention, his drooling was decreased, his tendency of hitting his face with his hands decreased. In the 3.sup.rd month, the tightness around hip joints decreased. His lateral cruising was better in the 6.sup.th month of administration of the formulation and he started showing inclination for academics.

Patient 2: A 7 year old girl was suffering from microcephaly with post-meningitis mental retardation, delayed speech and autistic features. She was brought with complaints of decreased sleep, self-injurious and self-stimulatory behavior, irritability, shouting, not asking for her needs and had no bladder or bowel control. With 2 months of treatment with the present invention, her sleep improved and her head circumference increased by 1 cm. Further, her understanding and chewing improved. After 4 months of treatment, sleep improved further, her feeding skills improved, and she started eating chapatti (Indian bread) when given in pieces. Her self-injurious tendency was reduced. At the 6.sup.th month, her eye contact was better and she started feeding herself independently most of the times. At the 10.sup.th month, she started speaking non-meaningful words and started going out of the house when she had the urge to do so. With 10 months of treatment, her vocalization increased substantially and comparatively she was less irritable and had started following few of her mother's commands.

Patient 3: A 9 year old female patient was suffering from microcephaly with mental retardation and delayed milestones. There was a history of delayed birth cry with low birth weight of 1.2 kg. Her Electro Encephelograph (EEG) showed evidence of bilateral centro-temporal epileptiform activity, right more than left. But she was not given any anti-seizure medication till then. She came with complaints of less balance while walking, speech was limited to the word “abba” (Indian word for father) only. She started responding significantly in only 1 month—her understanding improved, drooling was decreased, frequency of fall reduced, her head circumference increased from 43.8 to 44 cm. With 3 months of medication, her balance improved further, she started climbing a few steps without support, started eating comparatively more tidily, with better chewing of food and started spitting out liquids while brushing her teeth. With 6 months of medication, she showed global improvements in motor functions and cognitive functions—she started understanding a local language Marathi which she did not understand before, and she performed a good dance at school, stopped soiling her clothes, started to understand the difference between her belongings and those of others.

Patient 4: A 6 year old female patient was suffering from Attention Deficit Hyperactivity Disorder (ADHD). There was a family history of consanguineous marriage. She was brought to the clinic with complaints of mental subnormality and hyperactivity. She started responding in just 1 month of treatment with improved understanding and started following commands in a better way. She discontinued treatment after the 1.sup.st month for 4 months due to a cough and cold infection. Despite discontinuation of the treatment, her improvements continued in her tooth-brushing skills and she developed color concepts. With two more months of treatment, her understanding improved further, she started identifying most of the pictures in books and started writing a few alphabets in Arabic script with a good finger-grip. But her hyperactivity continued as before.

Patient 5: An 8 year old male patient coming from a lower-income group was suffering from Microcephaly, Attention Deficit Hyperactivity Disorder (ADHD) and delayed speech and language. The patient, also had behavioral problems. With just 1 month of treatment, his hyperactivity was reduced, started speaking many new words, starting calling his own name, his understanding was better and he was less irritable. With 3 months of treatment, his hyperactivity was further reduced, speech was still better with more clarity, he was more independent in his dressing abilities, and his understanding was further better. With 5 months of treatment, his head circumference was found to have increased from 45.5 to 45.7 cm. He started speaking at 2-3 word level sentences.

Patient 6: A 9 year old male patient came with complaints of Attention Deficit Hyperactivity Disorder (ADHD), delayed speech and with a few autistic features. He was obsessive about playing with plastic articles such as bags, etc. There was a history of consanguineous marriage. Birth history was normal, but at the age of 6-8 months, there was an incidence of fall from his bed and then jerks started followed by regression in milestones. Scan showed periventricular calcification. EEG showed right centro-parietal spikes. With 4 months of treatment his hyperactivity was less, he started sitting in one place for 15-20 minutes, obsession with polythene was reduced, he started obeying some orders and started attempting to dress himself. He started to inform parents about his toilet needs. With 5 months of treatment, his facial expressions were better, previous improvements were maintained, he started brushing his teeth and taking bath. With 10 months of treatment, his previous improvements were maintained and he started obeying simple requests like bringing a glass of water, bringing the TV remote control, etc.

Patient 7: A 15 year old male patient was diagnosed with cerebral palsy spastic diplegia. There was a history of premature delivery at 7 months, with low birth weight of 900 gms. There was a history of neonatal jaundice. He came with complaints of unable to walk effectively and reduced fine-motor skills. With just 2 months of medicine, his kneel-standing balance improved. With 4 months treatment his bilateral hip extension improved and with 7 months of treatment, slightly better standing tolerance was seen.

Patient 8: A 4 year old female patient was diagnosed with right hemiplegic cerebral palsy. She had a normal birth history. When she arrived for treatment, her speech was unclear and right-sided movement was not upto the mark. With 1 month of treatment, her grip was better, and she was more cheerful. With 4 months of treatment, her spasticity was reduced, her walking was better, hand functions were better. At the end of the 5.sup.th month, her physiotherapist remarked she has been doing very well in all aspects, has started stepping on her own on to a 7-inch high stool”.

Patient 9: A 14 year old female patient, was diagnosed with microcephaly with seizure disorder, right hemiparesis and mental subnormality. The birth history was full-term delivery with delayed birth cry and low birth weight. She had jaundice after birth and from the 3.sup.rd month onwards she had convulsions. She still gets convulsions at a gap of 2-3 months. Her scan showed mild cerebral atrophy and Electro Encephelograph (EEG) was abnormal. She came with complaints of delayed motor milestones, poor scholastic performance with right-sided weakness. With 6 months of treatment, she could make use of her right hand and could hold and release her grip. Her understanding was improved, head circumference even at this age increased from 46 to 47 cms. Her myoclonic jerks were less than before.

Patient 10: A 4 year old male patient was diagnosed with macrocephaly with hypotonia and global developmental delay. The birth history was full-term delivery, lower caesarian section (LCSC) done due to large head. Birth cry was normal. There is a history of neonatal jaundice. He was well upto 3 months and then started getting myoclonic jerks. By the 6.sup.th month, he started losing his eye-contact and social smile. Magnetic Resonance Imaging (MRI) showed dilated ventricles and EEG was abnormal. Metabolic screening tests were normal. He came with complaints of delayed motor, speech and mental milestones, seizure disorders and there was no bladder-bowel control. His parents have reported, that 1 week after starting the medicine, his limb movements increased, his looks and expressions became more meaningful and “trying to be happy and enjoying all his time. He is expecting pampering and people to talk to him and attend him”. With 5 months of treatment, he became more active, started recognizing parents and grandparents, making continuous noises, visual tracking of objects improved, sitting balance became better when made to sit. His supported-standing balance was slightly better and he was better emotionally and understanding improved.

Patient 11: A 57 year old male patient came with a history of numbness in the area of radial half—palmer aspect of hand, weakness of right arm, joint deformity of right index finger with diminished grip strength in both arms. The problem started about 2.5 years back with right shoulder pain. On investigation, MRI of cervical region showed cervical extensive spondiloarthopathy showing: 1) C5/6 level bilateral foraminal stenosis due to uncovertebral osteophytes, compression of right exiting C6 and bilateral exiting C7 nerve roots as well as diffuse posterior disk osteophyte complexes causing degenerative central canal stenosis at these levels. 2) No cervical compressive myelopathy seen. He was operated in December 2005 for decompression. Before starting treatment with the present invention his nerve conduction velocity (NCV) study showed evidence of motor nerve degeneration in muscles of both upper limbs (more severe on the right) at root, anterior horn cell level. With 4 months of treatment, his pain in the right index finger was less. With 6 months treatment, his repeat NCV study showed the motor activities in biceps and dorsal interossi showed improvement. With 9 months of treatment, his grip strength was much better. Considering his diagnosis of anterior horn cell level lesion, these improvements with 6 months of treatment are significant and shows objective evidence of regeneration with NCV study.

Patient 12: A 3 year 7 months old male patient was diagnosed withcame with a diagnosis of Down Syndrome. The main complaints included delayed speech milestones, congenital heart disease, recurrent cold cough and consistent leakage of urine drop by drop. Birth history included normal delivery (2.sup.nd child) with low birth weight (1.5 kg), 2D-echo test was suggestive of large ventricular septal defect (VSD), and there was delayed achievement of milestones (walking at 3 years). With 2 months of treatment, he started speaking words like “mummy” and “papa” and speaking letters like “A, B, C” and some Hindi alphabets in school. After 4 months of treatment, he achieved better walking balance and also tried to run. There was also a 60% improvement in understanding skills. After 15 months of treatment, his frequency of cold and cough reduced. Vocabulary of words improved along with increased clarity. His social interaction skills also improved.

Patient 13: A 2 year 3 months old female patient came with a diagnosis of hypotonic cerebral palsy with development delay. The main complaints included delayed motor and speech milestones. Birth history consisted of neonatal jaundice, delayed achievement of early motor milestones with sitting achieved at one year of age, and a normal Electro-encephalograph (EEG). With 1 month of treatment she started standing without support for few seconds. There was better standing tolerance with support (30 minutes) and better non-verbal communication and understanding skills. She also started enacting nursery rhymes and recognizing pictures cards. After 3 months of treatment, she started walking around four to five steps on her own and indicating her toilet needs. She also developed better verbal and nonverbal communication skills. After 5 months of treatment she started walking around ten to fifteen steps without support with a wide-based gait and easily maneuvering herself from sitting to standing position. She also started speaking two to three word sentences and recognizing all body parts. With 8 months of treatment, she started getting up from squatting position independently and started doing brushing, bathing and eating with minimal help. Her head circumference has increased from 47 to 48 cm.

Patient 14: A 4 year 9 months old male patient came with a diagnosis of microcephaly with cerebral palsy and spastic quadriplegia. The main complaints included delayed motor, speech and mental milestones. He also had breath-holding spells and only partial bowel and bladder control. Birth history included mother had typhoid during pregnancy and he experienced birth asphyxia with meconiun poisoning and neonatal seizures. After 1 months of treatment, his breath-holding spells reduced and he achieved better sitting balance (when made to sit). After 4 months of treatment, breath holding spells were further reduced. Head size increased from 45.5 to 46 cm. There was reduced tightness in upper extremities, better understanding skills, and a better sitting balance

Patient 15: An 11 months old female patient came with a diagnosis of microcephaly with epilepsy and global developmental delay. The main complaints included regular seizures, delayed motor, speech and mental milestones, cortical visual deficit, and difficulty engulfing food. At birth she was the 2.sup.nd of the twins and had birth asphyxia and low birth weight (1.4 kg). After 1 month of treatment she became less irritable. With 4 months of treatment, she experienced reduced jerks and a more neutral neck. Improvements noted with 5 months of treatment included increased head size (37 to 37.5 cm), better neck control, better weight gain, and more vocalization. With 6 months of treatment, improvements included better mental responses, better prone pushups, and better neck control. With 9 months of treatment, she had better sleep, better weight bearing on lower limbs, and started attempting to turn from supine to prone. With 16 months of treatment, she had even better neck holding, started responding to her name, had better facial expressions, and experienced reduced seizures.

Patient 16: A 19 year old male patient, came with a diagnosis of herido-familial mental retardation with conduct disorder. The main complaints included less understanding skills, poor reading and writing skills, and nail chewing. He had a birth history of consanguineous marriage of parents and two elder sisters had a similar presentation. With 1 month of treatment, he became more disciplined. With 2 months of treatment, he had better sleep and became quieter.

Patient 17: A 7 year 6 months old male patient came with a diagnosis of autistic features with attention deficit hyperactivity disorder. The chief complaints included hyperactivity, delayed speech milestones, seizure disorder, and over-eating habits. At birth, there was premature delivery (35-36 weeks) with low birth weight (1.52 kg) and neonatal convulsion. Computerized Tomography (CT) scan was normal and EEG was also normal. With 3 months of treatment he started to speak more. With 4 months of treatment, he started to speak new words and over-eating tendency was reduced.

Patient 18: A 10 year old male patient, came with a diagnosis of post meningo-encephalitic quadriplegia with extra-pyramidal symptoms. His main complaints included that he was unable to sit, stand and walk, there was regression of speech milestones, and he had no bladder/bowel control. Birth history was that he was normal till 7 years old. There was a history of chicken pox, vomiting and headache, followed by viral encephalitis (septic shock, acute respiratory distress syndrome—ARDS, hemorrhagic thalami, hippocampus). Improvements noted with 1 month of treatment include better understanding, he tried to speak small sentences, and there was better supported sitting balance. With 3 months of treatment there was better clarity of speech, better chewing of food, and he tried to get up from prone. Improvements with 10 months of treatment include he was able to sit independently from side line, held on to quadruped position, there was better hand to month coordination. He was able to stand against the wall and showed emotional expressions. There was better dietary intake and better understanding skills.

Patient 19: A 5 year 4 months old male patient came with a diagnosis of dysmorphic syndrome with seizure disorder and developmental delay. The chief complaints included delayed motor, speech and mental milestones, there was no bladder/bowel control, there was left sided weakness and seizures disorder. He was born by C-section with low birth weight (2.3 kg) with hypoglycemic convulsions. EEG was suggestive of underlying structural disease. Improvements noted with 4 months of treatment include reduced intensity of seizures. There was better supported standing balance and better bladder control. More babbling was observed along with better understanding skills. With 6 months of treatment there was better food intake, better eye contact, even more babbling and he started following commands.

Patient 20: A 3 years 6 months old female patient came with a diagnosis of microcephaly with dystonic quadriplegia and seizure disorder. The main complaints included delayed motor, speech and mental milestones, no bladder/bowel control, impaired vision, and seizure disorder. Birth history is that she was the 1.sup.st child, birth was pre-term (18 days prior) by emergency C-section. There was birth anoxia and low birth weight of 1.8 kg. CT scan brain was suggestive of encephalomalacia in parieto-occipital region. EEG was suggestive of epileptiform focus in left fronto-temporal regions. With 2 months of treatment she started indicating for bladder/bowel via sound/gestures. There was better visual tracking and better weight bearing on forearms in prone lying. With 4 months of treatment, there was less drooling. She started indicating her needs through gestures. There were better mental responses and better orientation to surroundings.

Patient 21: A 9 year 7 months old male patient came with a diagnosis of hypotonic cerebral palsy with herido-familial mental retardation. The main complaints were that he was unable to stand and walk independently and had poor academic skills. His parents had a consanguineous marriage with positive family history of developmental delay. He had a large skull since birth. There was a history of convulsion since 7 months of age. CT scan was suggestive of severe fronto-temporal atrophy. With 2 months of treatment he was able to crawl with support and cruised along the side the bed more frequently. He started communicating effectively at full sentences level and achieved intact bowel-bladder control. With 8 months of treatment, there was better muscle tone throughout and he achieved normal communication skills and better understanding skills. He started to frequently cruise alongside the bed and started trying to dress himself. With 11 months of treatment, muscle tone improved throughout. He started coming independently to supported standing position and was able to stand momentarily without support. Communication skills improved further and he could feed himself independently now. He was also able to put on his shirt and pants independently. With 16 months of treatment he was able to stand without support for little duration and was able to stand for half an hour at a stretch. He also achieved better understanding skills and there was an increase in his calf-girth (6.8 to 7.1 inches).

Patient 22: A 5 year old female patient came with a diagnosis of microcephaly with diplegic cerebral palsy with attention deficit hyperactivity disorder (ADHD). The main complaints included an imbalance while walking, unclear speech, and she was hyperactive. She was the 2.sup.nd child (elder sister 17 years old, normal). There was a history of convulsion at 5 months of age. Post-tibial somatosensory evoked potentials (SSEP) study was suggestive of left more than right disturbance in somato-sensory conduction in central somato-sensory pathway. EEG was suggestive of epileptiform abnormalities over the right occipital region. With 2 months of treatment she became less hyperactive. She could stand readily from lying down/sitting position and speak longer sentences. She developed better handwriting skills and could walk in a straight line. She could also do buttoning-unbuttoning now. Improvements noted with 4 months of treatment included better walking balance. She could climb stairs up and down without support and started writing “A, B, C”, and “1, 2, 3”. Improvements noted with 20 months of treatment include better school performance.

Patient 23: A 5 year old male patient came with a diagnosis of cerebral palsy with spastic diplegia. The main complaints included delayed motor milestones and unclear speech. He had a premature birth (30-32 weeks). There was low birth weight of 1.5 kgs. He had a history of respiratory distress syndrome with neonatal jaundice and 12 days of Neonatal Intensive Care Unit (NICU) care. There was delayed achievement of milestones (sitting 1.5 years). Improvements with 2 months of treatment include better balance and more clarity in speech. Improvements with 6 months of treatment include better understanding skills, better imagination and thinking, and more improved balance.

Patient 24: A 9 year male patient came with a diagnosis of athetoid cerebral palsy with right sided hemiplegia. The main complaints included delayed motor milestones, unclear speech, involuntary movements, and right sided weakness. He had a birth history of forceps delivery (cord around the neck) with birth anoxia and neonatal convulsions with jaundice. MRI was suggestive of putaminal gliotic lesions. Improvements with 5 months of treatment include better sitting balance, and better walking with rollator/walker. He had clearer speech and started standing without support for 1 to 2 minutes. There was also better speed of food intake. Improvements with 7 months of treatment include better sitting and standing balance and he could stand up till 120 to 130 counting. Improvements with 16 months of treatment include less involuntary movement, better sitting and standing balance, and he was able to switch the TV on and off.

Patient 25: A 4 year old male patient came with a diagnosis of global developmental delay with propionic academia. The main complaints included delayed motor and speech milestones, no bladder bowel control, less eye contact, and frequent mouthing. He was born by C-section (Breech position). There was normal development till four months of age, since then development was slow (sitting 1.5 years). He was found to be suffering from a metabolic disorder (propionic academia). There was normal hearing sensitivity and normal Visual Evoked Potential (VEP). MRI was suggestive of white matter hyper intensities and EEG was suggestive of multi focal epileptic focus. Improvements with 5 months of treatment include he became able to sit on his own and tried to balance with his elbows. He also tried to pick up eatables and feed himself and tried to attain crawling position. There was increased cooing, and he started saying words like “Appa”.

Patient 26: A 4 year old female patient came with a diagnosis of autism spectrum disorder with delayed speech and language milestones. The main complaints included disturbed sleep, she was constipated, was unable to walk well, no spontaneous speech, and was lost in her own world. Birth history includes she was 2.sup.nd child (elder sister normal). There was forceps delivery (breech presentation) with birth anoxia and neonatal convulsions with tracheostomy and 15 days of NICU care. She was operated for VP shunting at 3.sup.rd month of age, shunt was operated again at 9.sup.th months of age (infection). Improvements with 2 months of treatment include she started coming down the bed for passing urine. Was able to associate two words while speaking, and imitate lots of new words. She developed better orientation of people, better understanding skills, and better walking balance. With 4 months of treatment she developed better following of commands and better walking balance.

Patient 27: A 1 year 5 months old male patient came with a diagnosis of post meningitis developmental delay. The main complaints included delayed motor milestones, cortical visual deficit, right sided weakness, and right side hearing loss. Birth history consists of neonatal convulsion on 10.sup.th day of birth, and again at 1.5 month of age. CT scan was suggestive of pyogenic meningitis with cerebral oedema. MRI was suggestive of delayed myelination, while brainstem evoked response audiometry (BERA) was suggestive of right sided hearing loss. Improvements noted with 3.5 months of treatment include he could come independently to sitting position from supine line, started moving by bottom shifting, head size increased from 41.5 to 42 cms, started trying to stand with support, and was able to turn prone. With 5 months of treatment he developed better eye tracking skills, better standing balance, more words-babbling, better understanding skills, his head size increased from 42 to 42.5 cms, and he started moving by shifting chairs in front. Improvements with 6 months of treatment include better eye contact now, was able to take out pen from dad's pocket, started playing with toys meaningfully, developed better social interaction, started following more commands, more vocalization, and started trying to stand up from the floor. Improvements with 7 months of treatment include still better eye contact, started approaching objects regularly with both hands, was able to cruise along the furniture, developed better understanding skills, started pointing to a few body parts, and started speaking more words. Improvements noted with 12 months of treatment include still better visual tracking skills, was able to come independently to quadruped position and to supported standing position, and he developed better understanding skills.

Patient 28: A 15 year old male patient came with a diagnosis of attention deficit hyperactive disorder. The main complaints included less understanding skills, delayed language milestones, and he used to get angry frequently (occasionally violent). Birth history included premature birth (7.sup.th month of pregnancy) with low birth weight and delayed achievement of all milestones (walking at 4 years, speech at 8 years) together with positive dysmorphic features. With 2 months of treatment, he started trying to speak small sentences. Improvements with 3 months of treatment include less tendency of getting irritated, reduced anger, better behavior, started to watch “Animal Kingdom” channel on TV (earlier used to show no interest in TV). Improvements with 7 months of treatment include less spells of irritation, and while feeding he started saying when he was finished.

Patient 29: A 51 year old female patient came with a diagnosis of cerebral palsy with spastic quadriplegia with impaired speech. The main complaints included delayed physical and speech milestones. Improvements with 18 months of treatment include better painting skills with right hand, was able to manage eating with right hand with partial independency, and had better sleep and dietary intake.

Summary

No. of patients: 29; Male: 18; Female: 11; Age range: 1 year 5 months-57 years

Test Results:

TABLE 1

| Improvement seen in following areas | No. of cases |
|---|---|
| Drooling | 3 |
| Spasticity | 4 |
| Behavior | 3 |
| Understanding | 22 |
| Sleep | 4 |
| Fine motor skills | 5 |
| Irritability | 6 |
| Speech and vocalization | 16 |
| Motor movements | 18 |
| Balance | 6 |
| Following commands (cognition) | 8 |
| Hyperactivity | 2 |
| Jerks | 3 |
| Increase in head circumference | 4 |
| Bladder-bowel control | 3 |
| Attention span | 1 |
| Independence in daily activities | 2 |
| Axonal regeneration (Nerve conduction | 1 |
| velocity study) | |
| Global improvements | 1 |

The results show that the present invention has beneficial effects on motor and higher mental functions and can significantly improve the quality of life of patients with neurological disorders.

Similarly, the present invention may have said action in treating animals and other living organisms with nervous system disorders.

### EXAMPLE 2

Following potentized formulations were used alternatively or in combination for treatment of patients suffering from various neurological disorders:

Formulation No. 1 (“Higher Potency Formulation”): 1M Arnica montana; 30 C Hypericum perforatum; 30 C Causticum; 200 C Hyoscyamus niger; 30 C Zincum phosphoricum; 30 C Natrium muriaticum; 200 C Calcarea phosphorica; 200 C Kalium phosphoricum; and 200 C Ferrum phosphoricum

Formulation No. 2 (“Lower Potency Formulation”): 30 C Arnica montana; 30 C Hypericum perforatum; 30 C Causticum; 30 C Hyoscyamus niger; 30 C Zincum phosphoricum; 30 C Natrium muriaticum; 30 C Calcarea phosphorica; 30 C Kalium phosphoricum; and 30 C Ferrum phosphoricum

The ingredients in each of the two embodiments of the formulation were mixed in approximately equal proportions, that is, approximately 1/9^{th} part of each ingredient was mixed homogeneously with 1/9^{th} part of each of the other ingredients, to get 1 part of the resulting mixture. The final preparation was then added to sucrose globules allowing the globules to soak the liquid, preferably until the globules were thoroughly moistened. The potentized formulations were prepared by serial dilution according to the standard homeopathic procedures.

As will be evident to the reader, the choice of the potency chosen, whether high or low, is dependent on whether the mental symptoms are more marked than the physical symptoms. While, potencies in the range of 30 C to 1M have been used in the two formulations shown above, it is theoretically conceivable that if one is faced with a case with significantly marked physical symptoms—e.g. a case of spasticity in the limbs with no mental dysfunctions—a lower potency,e.g. 6×, could be the ideal choice. Similarly, in cases of attention deficit hyperactive disorders without any accompanying physical symptoms, higher potencies such LM potencies may be the potencies of choice. Hence, the homeopathic formulation subject hereof is claimed for the entire range of potencies from tincture to LM potencies.

Also, since the individual ingredients are so dilute due to the potentization, it will not matter, if say, a slightly higher proportion of one particular ingredient is used compared to the other 8 ingredients—even in this case the treatment result would remain the same.

The resultant combination formulation of the present invention was administered to eleven patients suffering from various neurological disorders. The patients were clinically monitored wherever possible. The details of the results and the patients that were administered are presented below:

Patient 1 (Higher Potency Formulation used): This patient was reported on 7 Oct. 2011 at the age of 4 years and 8 months with a diagnosis of ADHD and dysarthria. His chief complaints of low eye contact, inability to jump and hop, low concentration, drooling and restlessness and unclear speech. Birth history was suggestive of delay in achievement of all available milestones and he walked by age 17 months and speech was at 2.5 years. Improvements noted with 3 years of treatment using the higher potency formulation disclosed above included better eye contact, better sitting tolerance, better communication skills and making longer sentences, less sensory issues, better understanding skills, better response to questions, less echololia, better social understanding skills, less hyperactivity, better memory skills, better sitting tolerance in classroom and became much more independent in activities of daily living.

Patient 2 (Lower Potency Formulation used): This patient was reported on 17 Dec. 2012 at the age of 6 years with chief complaints of delayed motor milestones along with Kyphotic spine and deformities at knees. Birth history was suggestive of consanguineous marriage of parents and his elder sister reported a history of seizures. He was clinically diagnosed as a case of spinal muscle atrophy and sensory-motor neuropathy. His Electromyography/Nerve Conduction Velocity study was suggestive of neuropathy in both upper and lower extremities suggesting lower motor neuron disease. Improvements noted after 1 month of treatment with the Lower Potency Formulation disclosed hereinabove include better weight bearing on upper limbs and lower limbs was noticed. After 1 year of treatment with the Lower Potency Formulation disclosed hereinabove he became physically more active and started crawling for mobility whereas as before treatment he used to move by bottom shifting only.

Patient 3 (Lower Potency Formulation used): This patient was reported on 2 Aug. 2014 at the age of 29 years with a diagnosis of stroke and left hemiplegia. Her medical history was she had suffered a road accident on 10 Oct. 2013 and had been operated for craniotomy; her CT scan (17 Jun. 2014) was suggestive of changes of encaphalomalacia in the left fronto parietal lobe. Improvements noted after just 15 days of treatment with the Lower Potency Formulation disclosed hereinabove included she could identify people and relatives around, she became more expressive, she started opening up her jaws to take semi-solid diets and tears were observed when she was crying. After around one month of treatment, she became more expressive, started showing more emotional responses, there was less tightness on right hand and right leg, she could move her right half better, and there was better neck control. After around one year of treatment, she tried to utter more words and got better pain perception

Patient 4 (Lower Potency Formulation used): This patient was reported on 28 Jul. 2010 at the age of 43 years with a diagnosis of post-epidermoid tumor hydrocephalus with cerebral ataxia. Chief complaints included imbalance while walking, labored speech, less dietary intake, left sided weakness. Improvements after 4 years of treatment with the Lower Potency Formulation disclosed hereinabove included better balance in walking, is able to walk with a walker, better use of hands to operate keyboard, better dietary intake, better sleep, more erect posture, and better strength in left hand.

Patient 5 (Lower Potency Formulation used): This patient was reported on 7 Mar. 2014 at the age of 19 months with a diagnosis of Dysmorphic Syndrome and Global Developmental Delay. Chief complaints included delayed motor and speech milestones, constipation and lacking in bladder bowel indications. Improvements noted after 11 months of treatment with the Lower Potency Formulation disclosed hereinabove included less constipation, better chewing of food, better supported walking, better grip of objects, better exploration of surroundings and increase in head circumference from 44-45 cm.

Patient 6 (Lower Potency Formulation used): This patient was reported on 3 Sep. 2010 at the age of 3 years and 3 months and with a diagnosis of Diplegic Cerebral Palsy. His birth history was birth asphyxia and was given oxygen and MRI suggested delayed myelination. He reported to us with chief complaints of delayed motor milestones and unclear speech and frequent drooling. Improvements seen with 3 years of treatment with the Lower Potency Formulation disclosed hereinabove included that he was able to get up on his own from the floor and walk some distance though he was still lacking heel strike, less spasticity was seen in all the limbs, he started climbing staircases too, developed near normal communication skills, saw good cognitive improvement, started attending a normal school, developed good hand-writing skills and developed good social behavior.

Patient 7 (Lower Potency Formulation used): This patient was reported on 11 Jan. 2010 at the age of 3 years with a diagnosis of Down Syndrome and Developmental Delay. Her chief complaints were inability to walk independently and limited speech and constipation. Birth history was suggestive of neonatal jaundice. Improvements noted after 5 years of treatment with the Lower Potency Formulation disclosed hereinabove included she is able to walk independently now with a wide-based gait, better understanding skills, she can now indicate for her bladder bowel needs, less drooling is reported, she has started imitating words and has lesser sensory issues.

Patient 8 (Higher Potency Formulation used): This patient was reported on 6 Jan. 2014 at the age of 3 years and 9 months with a diagnosis of autism and delayed language and social milestones. She came with chief complaints of delayed language and social milestones and poor social interaction skills with limited eye contact. Birth history was suggestive of neonatal jaundice. Improvements seen after 7 months of treatment with the Higher Potency Formulation disclosed hereinabove include less auditory hypersensitivity reported, positive changes in social behavior and comparatively better eye-contact.

Patient 9 (Higher Potency Formulation used): This patient was reported on 23 Nov. 2011 at the age of 7 years with a diagnosis of Attention Deficit Hyperactivity Disorder (ADHD) and delayed language milestones. His chief complaints were hyperactivity and limited communication skills with difficulty in performing fine motor skills. Improvements seen with 3 years of treatment with the Higher Potency Formulation disclosed hereinabove include better communication skills and speaks new words now, better social behavior, better comprehension skills, improvements in motor skills, less constipation and feeds by himself.

Patient 10 (Lower Potency Formulation used): This patient was reported on 12 Oct. 2013 with a diagnosis of Subacute Sclerosing Panencephalitis (SSPE)—a potentially fatal disease. Her chief complaints were myoclonic jerks along with an inability to stand and walk since the last one month along with an inability to speak since the last 15 days. Along with that a difficulty in swallowing was also reported. After 1 month of treatment with the Lower Potency Formulation disclosed hereinabove, she started walking few steps without support and needed less external support to walk, there was reduced difficulty in swallowing or chewing, though myoclonic jerks were still reported. After 2 months of treatment with the Lower Potency Formulation disclosed hereinabove , reduction in frequency of myoclonic jerks was noted, she was walking but with a lot of swaying, speech was gradually improving, and better dietary intake was reported. After 5 months of treatment with the Lower Potency Formulation disclosed hereinabove , EEG was showing no significant change, clinically the jerks were seen reducing, better communication was also noted and improvement in gait was noted. After 8 months of treatment with the Lower Potency Formulation disclosed hereinabove , EEG still remained the same, myoclonic jerks had completely stopped, she had much better understanding skills, dietary intake was better and she started demanding special diet and gait was much improved. After 11 months of treatment, a normal EEG was reported, with a complete regression of periodic complexes and no evidence of epilelptiform activity, no clinical jerks reported, gait had further improved, better communication skills were noted, she started identifying colors, was able to walk on her own and showed much better sleep. EEG coming to normal shows objective evidence of the efficacy of treatment with the Lower Potency Formulation disclosed hereinabove in a potentially lethal disease.

Patient 11 (Lower Potency Formulation used): This patient was reported on 16 Jul. 2009 with a diagnosis of SubacuteSclerosingPanencephalitis (SSPE). His chief complaints were frequent myoclonic jerks along with an inability to sit, stand and walk, poor orientation and less of speech along with no bladder bowel indications. He was apparently normal till January 2008 when he reported a history of giddiness along with inability to balance while walking, and myoclonic jerk were reported. EEG was done on Jun. 7, 2009 was suggestive of gross abnormality and bursts suppression patterns with periodic complexes at 4-5 second and abnormal background, which is typical of SSPE. Cerebral Spinal Fluid (CSF) test for measles antibodies was also positive. Improvements noted with 5 years of treatment with the Lower Potency Formulation disclosed hereinabove include no clinical jerks noticed now, better mental responses reported when called upon, became more aware of surroundings, better neck control developed, better gross movements of right hand noted, better social smile, now takes food of his choice only, calls out to his mother, and less rigidity of limbs noted. EEG done on 28 Apr. 2014 is suggestive of no periodic complexes now and improvement in the background activity.The EEG report before and after treatment with the Lower Potency Formulation disclosed hereinabove is shown below. EEG report came to normal and this EEG report shows objective evidence of the efficacy of treatment with the Lower Potency Formulation disclosed hereinabove in a potentially lethal disease.

### EXAMPLE 3

Higher Potency Formulation disclosed hereinabove was used for treatment of 45 patients of Autism. Starting in the year 2009, the results of said treatment are listed in Table 2, as under:

TABLE 2

| | Social | | Speech and | |
|---|---|---|---|---|
| Particulars | Behavior | Cognition | Communication | Hyperactivity |
| Cases | 29 | 33 | 26 | 23 |
| Improved | | | | |
| No Change | 15 | 12 | 19 | 18 |
| Observation | 1 | 0 | 0 | 4 |
| Not Made | | | | |
| Total Cases | 45 | 45 | 45 | 45 |

Technical Advancement and Economic Significance

The present invention offers several technical advancements and economic significance as given below:

The homeopathic formulation of the present invention can be effective in healing several types of neurological disorders including, but not limited to Cerebral Palsy—spastic, hyptonic, athetoid, ataxic; Mental Subnormality; Depression arising from neurological disorders; Autism; Down Syndrome; Global Delays; Developmental Disabilities; Neuropathies; Brain Injuries; Neurometabolic Disorders; Stroke; Attention deficit disorder (ADD); Attention deficit hyperactivity disorder (ADHD); Apert's Syndrome; Congenital Brain Anomalies like Schizencephaly, Pachygyria, Porencephaly; Dystonias; Herido-Familial Mental Retardation; Infantile Hemiplegia; Kernicterus; Learning Disabilities; Multi-Infarct Dementias; Microcephaly; Metabolic Disorders with Neurological Complications; Post Meningo-Encephalitic Brain Damage; Rubinstein Taybi Syndrome; Retts Syndrome; Post Surgical Neurodeficit; Multiple Sclerosis; Subacute sclerosing panencephalitis (SSPE) and various Syndromes and conditions of developmental delay and neurological conditions like Leigh's Encephalopathy, Cornelia De Lange Syndrome, Japanese Encephalitis, Tuberous Sclerosis, Leukodystrophy, Chromosomal Anamolies, Agenesis of Corpus Callosum, and Spinocerebellar Syndrome.

The homeopathic formulation of the present invention is stable, non-toxic and free of any side effects.

The homeopathic formulation of the present invention is easy to prepare.

Further, the homeopathic formulation can be combined with other homeopathic preparations to achieve desirable effects.

The homeopathic formulation of the present invention is a common formulation for several neurological disorders, and no drug individualization using single drug ingredient per dose as practiced in classical homeopathy is required.

The disclosed homeopathic formulation is cost-effective and the treatment cost is also inexpensive.

While considerable emphasis has been placed herein on the various features of the preferred embodiment, it will be appreciated that many alterations can be made and that many modifications can be made in the preferred embodiment without departing from the principles of the invention. These and other changes in the preferred embodiment as well as other embodiments of the invention will be apparent to those skilled in the art from the disclosure herein, whereby it is to be distinctly understood that the foregoing descriptive matter is to be interpreted merely as illustrative of the invention and not as a limitation.

We claim:

1) A formulation for treatment of neurological disorders, comprising:
a) Arnica montana;
b) Hypericum perforatum;
c) Causticum;
d) Hyoscyamus niger;
e) Zincum phosphoricum;
f) Natrium muriaticum;
g) Calcarea phosphorica;
h) Kalium phosphoricum; and
i) Ferrum phosphoricum.
Wherein each ingredient has a potency ranging from tincture to all X, C and LM potencies and proportion of each ingredient ranges from 1:1 to 1:9 with respect to the other.

2) The formulation of claim 1, wherein said ingredients in said formulation have a potency ranging from 30 C to 1M.

3) The formulation of claim 1, wherein at least one of the ingredients is potentized and is in the form selected from a group comprising of tincture, ethanolic solution and aqueous extract.

4) The formulation of claim 1, wherein said formulation is potentized by serial dilution and succussion prior to formulation.

5) The formulation of claim 1, wherein each of the said ingredients is potentized by serial dilution and succussion prior to formulation.

6) The formulation of claim 1, wherein wherein the formulation further comprises physiologically acceptable carriers are selected from the group consisting of whey, sucrose, calcium carbonate, microcrystalline cellulose, carbon, carnauba wax, croscarmellose sodium, stearic acid, magnesium stearate, silicon dioxide and ethanol.

7) The formulation of claim 1, wherein the formulation is administered in a form selected from the group comprising tablets, capsules, powders, globules, lozenges, pills, pellets, solution, syrup, elixir, suspension and emulsion.

8) The formulation of claim 1, wherein the neurological disorder is selected from the group comprising neurological disorders are selected from the group consisting of Cerebral Palsy—spastic, hyptonic, athetoid, ataxic; Mental Subnormality; Depression arising from neurological disorders; Autism; Down Syndrome; Global Delays; Developmental Disabilities; Neuropathies; Brain Injuries; Neurometabolic Disorders; Stroke; Attention deficit disorder (ADD); Attention deficit hyperactivity disorder (ADHD); Apert's Syndrome; Congenital Brain Anomalies like Schizencephaly, Pachygyria, Porencephaly; Dystonias; Herido-Familial Mental Retardation; Infantile Hemiplegia; Kernicterus; Learning Disabilities; Multi-Infarct Dementias; Microcephaly; Metabolic Disorders with Neurological Complications; Post Meningo-Encephalitic Brain Damage; Rubinstein Taybi Syndrome; Retts Syndrome; Post Surgical Neurodeficit; Multiple Sclerosis; Subacute sclerosing panencephalitis (SSPE) and various Syndromes and conditions of developmental delay and neurological conditions like Leigh's Encephalopathy, Cornelia De Lange Syndrome, Japanese Encephalitis, Tuberous Sclerosis, Leukodystrophy, Chromosomal Anamolies, Agenesis of Corpus Callosum, and Spinocerebellar Syndrome.

9) A high potency medication for treatment of neurological disorders of claim 8, comprising:
a) 1M Arnica montana;
b) 30 C Hypericum perforatum;
c) 30 C Causticum;
d) 200 C Hyoscyamus niger;
e) 30 C Zincum phosphoricum;
f) 30 C Natrium muriaticum;
g) 200 C Calcarea phosphorica;
h) 200 C Kalium phosphoricum; and
i) 200 C Ferrum phosphoricum
Wherein each ingredient optionally has a potency ranging from tincture to all X, C and LM potencies and said ingredients are mixed in equal proportions equalling 1/9^{th} part of each ingredient being mixed homogeneously with 1/9^{th} part of each of the other ingredients, to get 1 part of the resulting mixture.

1) A low potency medication for treatment of neurological disorders of claim 8, comprising:
a) 30 C Arnica montana;
b) 30 C Hypericum perforatum;
c) 30 C Causticum;
d) 30 C Hyoscyamus niger;
e) 30 C Zincum phosphoricum;
f) 30 C Natrium muriaticum;
g) 30 C Calcarea phosphorica;
h) 30 C Kalium phosphoricum; and
i) 30 C Ferrum phosphoricum
Wherein each ingredient optionally has a potency ranging from tincture to all X, C and LM potencies and said ingredients are mixed in equal proportions equalling 1/9^{th} part of each ingredient being mixed homogeneously with 1/9^{th} part of each of the other ingredients, to get 1 part of the resulting mixture

11) The formulation of claim 1, wherein potency of the formulation to be administered for treatment of neurological disorders dependent on whether the mental symptoms are more marked than the physical symptoms.
